(19) United States
(12) Patent Application Publication
(10) Pub. No.: US 2024/0366428 A1
(43) Pub. Date: Nov. 7, 2024

(54) METHOD FOR PROVIDING CONTROL DATA FOR AN OPHTHAMOLOGICAL LASER OF A TREAMENT APPARATUS FOR CORRECTING AN ASTIGMATISM

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/651,998

(22) Filed: May 1, 2024

(30) Foreign Application Priority Data
May 4, 2023 (DE) 10 2023 111 634.7

(51) Int. Cl. A61F 9/008

(52) U.S. Cl. CPC A61F 9/008; A61F 2009/00844; A61F 2009/00872

(57) ABSTRACT

The invention relates to a method for providing control data for an ophthalmological laser (12) of a treatment apparatus (10) for correcting an astigmatism, comprising as steps determining (S10) first astigmatism values from predetermined visual disorder data of a patient, wherein the visual disorder data provides a measured refraction of a patient; determining (S12) second astigmatism values from predetermined measurement data of a cornea (16) of the patient, wherein the measurement data provides a morphology of the cornea (16); ascertaining (S14) astigmatism correction data for the astigmatism correction of the cornea (16), wherein the first and second astigmatism values are combined by a combination rule for ascertaining the astigmatism correction data; and providing (S16) the control data for the ophthalmological laser (12), which includes the ascertained astigmatism correction data.

### FIELD

The invention relates to a method for providing control data for an ophthalmological laser of a treatment apparatus for correcting an astigmatism. Furthermore, the invention relates to a control device, which is configured to perform the method, to a treatment apparatus with such a control device, to a computer program comprising commands, which cause the treatment apparatus to execute the method, and to a computer-readable medium, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can for example be formed such that laser pulses effect a photodisruption and/or ablation in a focus situated within the organic tissue, to remove a tissue, in particular a tissue lenticule, from the cornea.

A correction of an astigmatism can also be performed by these treatment apparatuses. In case of an astigmatism, the cornea of the eye is not uniformly curved in all planes, whereby blurred or distorted vision can occur. In the treatment of the astigmatism, however, there is disagreement, which data is used as a basis for the astigmatism correction. On the one hand, visual disorder data of a patient can be used, which provides a measured refraction, in particular a subjective refraction, of the cornea, for example with a sphere and cylinder value in diopters and an axis value in degrees, wherein the astigmatism correction can be planned based on these refraction values. On the other hand, measurement data of the cornea can be ascertained, in which a morphology, for example a topology of the cornea, can be ascertained, wherefrom astigmatic curvatures of the cornea can also be ascertained. In particular, it can occur that the astigmatism values, which can be ascertained from these two methods, differ from each other, wherein it is then questionable, on which data the astigmatism correction is to be based.

### SUMMARY

Therefore, it is the object of the invention to provide control data, in which an astigmatism correction is ascertained in improved manner.

This object is solved by the independent claims. Advantageous embodiments of the invention are disclosed in the embodiments presented herein, the following description, as well as the figures.

The invention is based on the idea that the astigmatism correction data is adapted to the best suitable combination of refractive astigmatism and a corneal toricity for the treatment with the ophthalmological laser. Hereto, combination rules are provided, by which astigmatism values of both methods are combined for ascertaining the astigmatism, to overall achieve a better treatment result. Herein, the combination rule, which is used, may depend on which astigmatism values are initially present.

An aspect of the invention relates to a method for providing control data for an ophthalmological laser of a treatment apparatus for correcting an astigmatism, wherein the method comprises the following steps performed by a control device. Determining first astigmatism values from predetermined visual disorder data of a patient, wherein the visual disorder data provides a measured refraction of a patient, determining second astigmatism values from predetermined measurement data of a cornea of the patient, wherein the measurement data provides a morphology of the cornea, and ascertaining astigmatism correction data for the astigmatism correction of the cornea, wherein the first and second astigmatism values are combined by a combination rule for ascertaining the astigmatism correction data, are effected. Finally, providing the control data for the ophthalmological laser, which includes the ascertained astigmatism correction data, is effected.

In other words, first astigmatism values are first determined from predetermined visual disorder data of a patient and second astigmatism values are determined from predetermined measurement data of the cornea of the patient with the aid of the control device. Therein, the visual disorder data can originate from a medical examination of the patient and specify refraction values, which describe the astigmatism. For example, they can be ascertained by a refractometer. In contrast, the second astigmatism values are determined from measurement data of the cornea, which means that a measurement of the corneal curvature or corneal topography herein occurs, from which the astigmatism can be ascertained. For example, this can be performed by a keratograph.

After the first astigmatism values, which are obtained from the refraction of the patient, and the second astigmatism values, which are obtained from the morphology of the cornea, are determined, the respective astigmatism values can be combined by a combination rule, which may be selected depending on the determined astigmatism, in particular depending on a difference of the first and second astigmatism values. The combination rule can be a calculation rule, which composes the astigmatism values, in particular components of the astigmatism values, to combined astigmatism values. Thus, an improved consideration of effects, which are determined by the refraction, and effects, which are determined by the morphology, can be provided. In a simple case, it can for example be provided that an astigmatism correction is completely based either on the first or the second astigmatism values. Alternatively, an average value of the first astigmatism values and second astigmatism values can for example be formed to obtain a compromise of both determination methods. However, in case of more complex application, a combination of the first and second astigmatism values can also be provided, wherein the combination type is depending on which difference between the astigmatism values is present. For example, the astigmatism values can be decomposed according to components, for example according to a component for an astigmatism according to the norm (cardinal astigmatism or astigmatism rectus) and an oblique astigmatism (astigmatism obliquus), wherein one of these components can be weighted higher according to used combination rule.

After the first and second astigmatism values have been combined by the combination rule to combined astigmatism values, an astigmatism correction can be planned based thereon, wherein it can be provided in the form of astigmatism correction data, by which the astigmatism can be corrected by the ophthalmological laser. This astigmatism correction data can then be provided in the form of control data for the ophthalmological laser and/or the treatment apparatus, wherein laser pulses can be placed in the cornea by the control data such that a correction according to the astigmatism correction data can be performed.

For determining the astigmatism correction data, it may first be provided that the respective astigmatism values are first normalized to a same refractive index and that they are normalized to a same reference plane in the cornea, since the corneal vertex serves as a reference plane for the cornea, but the refraction is usually defined in a glasses plane in front thereof or in the pupil plane in an aberrometer. In particular, the astigmatism values can all be normalized to a plane in the corneal vertex.

By the invention, the advantage arises that both data sources, from which the astigmatism values originate, can be considered by a combination of the first and second astigmatism values, which results in better treatment results.

The invention also includes embodiments, by which additional advantages arise.

According to an embodiment a difference between the first and second astigmatism values is determined, and wherein the combination rule is provided depending on the ascertained difference. In other words, multiple combination rules can be provided, from which a combination rule is selected depending on the difference of the first and second astigmatism values. Thus, in case of a smaller difference between the astigmatism values, another combination rule can for example be selected than in case of a great difference.

Therein, the combination rules can be preset and/or a calculation rule can be present, which has the difference as a variable. For example, a combination rule K, which can for example be provided as treatment cylinder vector, can be defined as:

$$K = {A}_{1} + F * ( {A}_{2} - {A}_{1} ) ,$$

wherein A₁ is the first astigmatism values, A₂ is the second astigmatism values and F is a factor, wherein the factor can be a value between 0 and 1, wherein the values of 1 and 0 may be excluded. By this embodiment, the advantage arises that depending on the difference between the astigmatism values, a combination rule matched thereto can be provided, by which the astigmatism correction improves.

In another embodiment the respective astigmatism values are divided into two vector components, wherein the first astigmatism values comprise a vector J_{0,R} for a cardinal astigmatism, in which cylinder axes of the astigmatism for 0 degrees and 90 degrees are perpendicular to each other, and a vector J_{45,R} for an oblique astigmatism, in which cylinder axes for 45 degrees and 135 degrees are perpendicular to each other, wherein the second astigmatism values comprise a corresponding vector J_{0,K} for the cardinal astigmatism and a corresponding vector J_{45,K} for the oblique astigmatism. This means that the respective astigmatism values can be expressed in the notation from J₀ and J₄₅, which originates from the clinical practice and describes a cylinder correction, which is ascertained by a Jackson cross cylinder. This notation was for example described by Thibos et al. (“Power Vectors: An Application of Furier Analysis to the Description and statistical Analysis of Refractive Error”). With the cardinal astigmatism of the vector J₀, the astigmatism according to or against the norm (astigmatism rectus/inversus or astigmatism curvitale) is meant, and with the vector J₄₅, the oblique astigmatism (astigmatism obliquus or astigmatism torsionale) is meant. Therein, the subscript R in the vector components JR stands for the vector components of the astigmatism values, which is ascertained from the refraction of the patient, and the subscript K of the vector component J_{K} stands for the vector component, which is obtained from the astigmatism values of the morphology of the cornea. In particular, the vector components can also be represented in the form J(α+0/α+90) and J(α+45/α+135). Alternatively, the astigmatism can also be converted into a matrix form, wherein an affine transformation by a transformation matrix, for example 5×5 matrix, can then be used. The representation of the respective astigmatism values as vector components, in particular as basic vectors, results in the advantage that suitable combination rules can be provided and thus portions of the respective astigmatism values can be selected in improved manner.

In another embodiment a first combination rule is provided as the combination rule, by which an average value between the first and second astigmatism values is determined. In particular, the first and second astigmatism values can be provided as vectors or vector components, wherein the average value is a center on a difference vector. Hereby, the advantage arises that a minimization of a remaining magnitude of a global astigmatism can be achieved.

In an advantageous embodiment, the first combination rule includes that the cardinal astigmatism is calculated by (J_{0,R}+J_{0,K})/2 and the oblique astigmatism by (J_{45,R}+J_{45,K})/2. From this combined cardinal astigmatism and combined oblique astigmatism, the astigmatism correction data for correcting the astigmatism can subsequently be determined. By this embodiment, an equidistant deviation between the respective vectors can be achieved, which results in a uniform consideration of the differently ascertained astigmatism values.

In particular, it is provided that the first combination rule is used if the difference between the first and second astigmatism values is below a preset threshold value. This means that if the difference between the first and second astigmatism values is small, the average value between the vector components can be used to determine the astigmatism correction data therefrom. In particular, the threshold value can be specified as an absolute or relative value. For example, the first combination rule can be used if the difference of the astigmatism values is less than 3 diopters and/or the difference is below 10 percent of one of the astigmatism values.

In another embodiment a second combination rule is provided as the combination rule, by which that one of the first and second astigmatism values identical in sign is selected, wherein the astigmatism value has a magnitude closer to zero. This means that the combined astigmatism is minimized to the size of the smaller astigmatism value. Hereby, the advantage arises that an overcorrection is avoided, whereby corneal tissue can be saved.

In an alternative embodiment, this one of the first and second astigmatism values identical in sign is selected as the second combination rule, which is greater than or farther away from 0.

In an advantageous embodiment, the second combination rule includes that the cardinal astigmatism is provided by a maximum of J_{0,R} and J_{0,K}, if both are less than 0, and by a minimum of J_{0,R} and J_{0,K}, if both are greater than 0, and by 0 for all further cases, and wherein the oblique astigmatism is provided by a maximum of J_{45,R} and J_{45,K}, if both are less than 0, and by a minimum of J_{45,R} and J_{45,K}, if both are greater than 0, and by 0 for all further cases. In other words, by the second combination rule, it can apply to combined astigmatism values of the cardinal astigmatism J₀ and the oblique astigmatism J₄₅:

$${J}_{0} = \begin{matrix}{J}_{0 , R} < 0 , {J}_{0 , K} < 0 \text{=>} max ( {J}_{0 , R} ; {J}_{0 , K} ) \\ {J}_{0 , R} > 0 , {J}_{0 , K} > 0 \text{=>} min ( {J}_{0 , R} ; {J}_{0 , K} ) \\ else 0\end{matrix}$$

$$and$$

$${J}_{45} = \begin{matrix}{J}_{45 , R} < 0 , {J}_{45 , K} < 0 \text{=>} max ( {J}_{45 , R} ; {J}_{45 , K} ) \\ {J}_{45 , R} > 0 , {J}_{45 , K} > 0 \text{=>} min ( {J}_{45 , R} ; {J}_{45 , K} ) \\ else 0\end{matrix}$$

wherein the minimized vector component is thus respectively adopted, which is closer to the value of zero.

In an alternative embodiment, the cardinal astigmatism is provided by a minimum of J_{0,R} and J_{0,K}, if both are less than 0, and by a maximum of J_{0,R} and J_{0,K}, if both are greater than 0, and by 0 for all further cases, and wherein the oblique astigmatism is provided by a minimum of J_{45,R} and J_{45,K}, if both are less than 0, and by a maximum of J_{45,R} and J_{45,K}, if both are greater than 0, and by 0 for all further cases.

In particular, it is provided that the second combination rule is used if the difference between the first and second astigmatism values is above a preset threshold value. In particular, the second combination rule can be used if a keratoconus is present in the cornea. The threshold value can for example be selected at a cylinder value of above 3 diopters. Herein, the advantage arises that the cornea is not overcorrected to save corneal volume, in particular for possible follow-up treatments.

In another embodiment a third combination rule is provided as the combination rule, by which the first and second astigmatism values are combined such that the cardinal astigmatism is considered as higher than the oblique astigmatism for the astigmatism correction. In other words, that component of the cardinal astigmatism, which is greater, can for example be selected from the first and second astigmatism values. For the oblique astigmatism, an average value of the components of the first and second astigmatism values can for example be formed.

In an alternative embodiment, the oblique astigmatism is considered as higher than the cardinal astigmatism.

In an advantageous embodiment, the third combination rule includes that the cardinal astigmatism is provided by a maximum of J_{0,R} and J_{0,K}, if both are less than 0, and by a minimum of J_{0,R} and J_{0,K}, if both are greater than 0, and by 0 for all further cases, and wherein the oblique astigmatism is calculated by (J_{45,R}+J_{45,K})/2. In other words, by the third combination rule, it can apply to combined astigmatism values of the cardinal astigmatism J₀ and the oblique astigmatism J₄₅:

$${J}_{0} = \begin{matrix}{J}_{0 , R} < 0 , {J}_{0 , K} < 0 \text{=>} max ( {J}_{0 , R} ; {J}_{0 , K} ) \\ {J}_{0 , R} > 0 , {J}_{0 , K} > 0 \text{=>} min ( {J}_{0 , R} ; {J}_{0 , K} ) \\ else 0\end{matrix}$$

$$and$$

$${J}_{45} = ( {J}_{45 , R} + {J}_{45 , K} ) / 2.$$

In an alternative embodiment, the cardinal astigmatism is provided by a minimum of J_{0,R} and J_{0,K}, if both are less than 0, and by a maximum of J_{0,R} and J_{0,K}, if both are greater than 0, and by 0 for all further cases, and wherein the oblique astigmatism is calculated by (J_{45,R}+J_{45,K})/2.

In particular, it is provided that the third combination rule is used if the difference between the first and second astigmatism values is above a preset first threshold value and it is further determined that a residual astigmatism above a preset second threshold value remains in the cornea after the treatment. In particular, this can occur in case of a keratoconus, in which a difference between the astigmatism values is great, in particular greater than 3 diopters, wherein the astigmatism cannot be completely eliminated by a treatment. This means that a value still remains as a residual astigmatism, which is above a preset second threshold value. The second threshold value can for example be at 0.5 diopters or greater. Hereby, the advantage arises that in particular those components of the astigmatism can be treated, which are usually considered more annoying for patients.

In another embodiment the refraction is determined by a refractometer and/or a phoropter and/or an aberrometer. In other words, the visual disorder data, from which the refraction and thus the first astigmatism values are determined, can be performed by measurements, which result in a statement about a vision of the patient. Herein, a refractometer and/or a phoropter and/or an aberrometer can for example be used.

In another embodiment the morphology of the cornea is determined by a topography device, in particular a keratograph, and/or a tomography device, in particular an optical coherence tomograph and/or a Scheimpflug tomograph. In other words, the measurement data, from which the morphology of the cornea and thus the second astigmatism values can be determined, can be based on topographic measurements of the cornea, whereby curvatures can be obtained, from which the second astigmatism values can be ascertained. Herein, devices for topography measurement, for example a keratograph, and/or devices for tomography measurement, in particular an optical coherence tomograph (OCT) and/or a Scheimpflug tomograph, can in particular be used.

A further aspect of the invention relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume with predefined interfaces of a human or animal eye by optical breakdown, in particular at least partially separate it by photodisruption and/or to ablate corneal layers by (photo)ablation and/or to effect a laser-induced refractive index change in the cornea and/or the eye lens and/or to increase a crosslinking of the cornea.

In a further advantageous embodiment of the treatment apparatus according to the invention, the laser can be suitable to emit laser pulses in a wavelength range between 300 nm and 1400 nm, for example between 900 nm and 1200 nm, at a respective pulse duration between 1 fs and 1 ns, for example between 10 fs and 10 ρs, and a repetition frequency of greater than 10 kilohertz (kHz), for example between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakdown can be spatially narrowly limited such that a high incision accuracy is allowed in the generation of the interfaces. In particular, the range between 700 nm and 780 nm can also be selected as the wavelength range.

In a further advantageous embodiment of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematic method diagram for providing control data according to an exemplary embodiment.

### DETAILED DESCRIPTION

In the figures, identical or functionally identical elements are provided with the same reference characters.

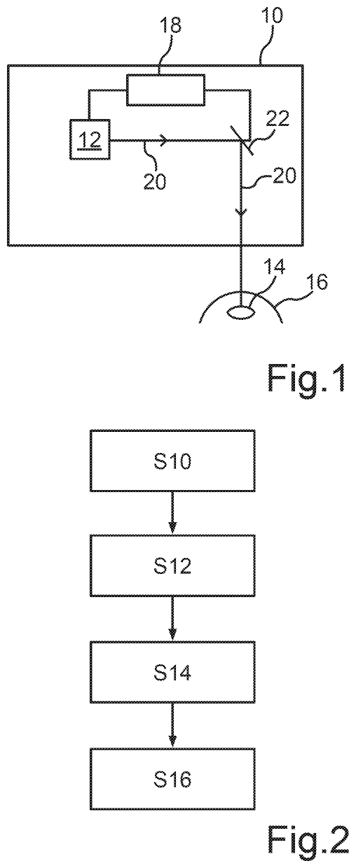

FIG. 1 shows a schematic representation of a treatment apparatus 10 with an ophthalmological laser 12 for removing a tissue 14 from a human or animal cornea 16 by photodisruption and/or ablation. For example, the tissue 14 can represent a lenticule or also volume body, which can be separated from the cornea 16 by the eye surgical laser 12 for correcting a visual disorder. In particular, an astigmatism of the cornea 16 can be corrected by removing the tissue 14, wherein astigmatism correction data can for example provide a geometry of the tissue 14 to be removed for the astigmatism correction. This astigmatism correction data can for example be ascertained by a control device 18 of the treatment apparatus 10 or by an external control device, which can for example belong to a planning device. Further, the control device 18 of the treatment apparatus can be formed to control the laser 12 of the treatment apparatus 10 by control data, which in particular includes the astigmatism correction data, such that it emits pulsed laser pulses in a pattern predefined by the control data into the cornea 16 of the eye to remove the tissue 14.

Furthermore, FIG. 1 shows that the laser beam 20 generated by the laser 12 can be deflected towards the cornea 16 by a beam deflection device 22 such as for example a rotation scanner, to remove the tissue 14. The beam deflection device 22 can also be controlled by the control device 18 to position the laser pulses of the laser beam 20 in the cornea 16 to remove the tissue 14.

The illustrated laser 12 may be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 fentoseconds and 10 picoseconds, and a repetition frequency of greater than 10 kilohertz, for example between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

In order to obtain the astigmatism correction data for correcting an astigmatism by the treatment apparatus, the method shown in FIG. 2 may be performed.

In FIG. 2, a schematically illustrated method diagram for a method for providing control data for an ophthalmological laser 12 of a treatment apparatus 10 for correcting an astigmatism is illustrated.

In a step S10, first astigmatism values can be determined from predetermined visual disorder data of a patient, wherein the visual disorder data can for example be ascertained by a refractometer and/or a phoropter and/or an aberrometer. Then, refraction values, which describe the astigmatism, can be determined from this visual disorder data.

In a step S12, second astigmatism values can be determined from measurement data, which has in particular been measured by a topography device and/or a tomography device.

For example, a keratograph can be used to determine a topography of the cornea, and/or an optical coherence tomograph can be used to create a tomography of the cornea. From this morphology, curvatures of the cornea can then be determined, by which the astigmatism can be specified in the form of the second astigmatism values.

In a step S14, the first astigmatism values and the second astigmatism values can be combined with each other by a combination rule to ascertain astigmatism correction data therefrom. In particular, the astigmatism correction data can serve to treat a determined astigmatism in the cornea of the patient by the laser 12. For example, multiple combination rules can be present, wherein the combination rule used for the combination may be determined depending on an ascertained difference between the first and second astigmatism values.

It is particularly preferred that the first and second astigmatism values are expressed in the form of vector components of a Jackson cross cylinder, wherein one of the component J₀ is here specified as a cardinal astigmatism, in which cylinder axes of 0 degrees and 90 degrees are perpendicular to each other, and a vector J₄₅ for an oblique astigmatism, in which cylinder axes of 45 degrees and 135 degrees are perpendicular to each other. Thus, a cardinal astigmatism (J_{0,R}) and an oblique astigmatism (J_{45,R}) can respectively be provided for the first astigmatism values, and a cardinal astigmatism (J_{0,K}) and the oblique astigmatism (J_{45,K}) can be correspondingly provided for the second astigmatism values.

If a difference between the first and second astigmatism values is below a preset threshold value, in particular below 3 diopters, a first combination rule can be used, by which an average value between the astigmatism values is provided. According to the vector component notation of the Jackson cross cylinder, it can in particular be provided that it applies to combined astigmatism values:

$${J}_{0} = ( {J}_{0 , R} + {J}_{0 , K} ) / 2 and {J}_{45} = ( {J}_{45 , R} + {J}_{45 , K} ) / 2.$$

In particular, it can be provided that in case of small differences between the respective astigmatism values, an average value of the vector components represents a good compromise, with which the astigmatism can be treated.

If the difference between the first and second astigmatism values is above a preset threshold value, in particular above 3 diopters, it can be provided to use a combination rule, in which corneal volume can be saved. For example, this can be present in case of a keratoconus. Herein, a second combination rule for combining the astigmatism values may be provided, wherein those astigmatism values, in particular those vector components of the respective astigmatism values with a magnitude closer to 0, are selected by the second combination rule. This means that with a vector component notation according to the Jackson cross cylinder, the following conditions can apply to combined astigmatism values:

$${J}_{0} = \begin{matrix}{J}_{0 , R} < 0 , {J}_{0 , K} < 0 \text{=>} max ( {J}_{0 , R} ; {J}_{0 , K} ) \\ {J}_{0 , R} > 0 , {J}_{0 , K} > 0 \text{=>} min ( {J}_{0 , R} ; {J}_{0 , K} ) \\ else 0\end{matrix}$$

$$and$$

$${J}_{45} = \begin{matrix}{J}_{45 , R} < 0 , {J}_{45 , K} < 0 \text{=>} max ( {J}_{45 , R} ; {J}_{45 , K} ) \\ {J}_{45 , R} > 0 , {J}_{45 , K} > 0 \text{=>} min ( {J}_{45 , R} ; {J}_{45 , K} ) \\ else 0\end{matrix}$$

Accordingly, an astigmatism correction can be performed without removing additional corneal volume.

If the difference of the first and second astigmatism values is above the preset first threshold value, which can for example be at 3 diopters, and it is further determined that the astigmatism cannot be completely removed by a treatment and a residual astigmatism remains, which is above a second threshold value, then, it can be provided that the cardinal astigmatism, in which cylinder axes of 0 degrees and 90 degrees are perpendicular to each other, is considered as higher than the oblique astigmatism. Expressed as vector components in the Jackson cross cylinder notation, this means that it may apply to the combined astigmatism:

$${J}_{0} = \begin{matrix}{J}_{0 , R} < 0 , {J}_{0 , K} < 0 \text{=>} max ( {J}_{0 , R} ; {J}_{0 , K} ) \\ {J}_{0 , R} > 0 , {J}_{0 , K} > 0 \text{=>} min ( {J}_{0 , R} ; {J}_{0 , K} ) \\ else 0\end{matrix}$$

$$and$$

$${J}_{45} = ( {J}_{45 , R} + {J}_{45 , K} ) / 2 .$$

Thus, it can be achieved that this component, which is usually perceived as more annoying in the astigmatism, is more severely corrected than the oblique component.

Finally, control data for the ophthalmological laser 12 can be provided in a step S16, which includes the ascertained astigmatism correction data. With this provided control data, the laser 12 and/or the treatment apparatus 10 can then be controlled for correcting the astigmatism.

Overall, the examples show how an astigmatism correction can be performed by vector planning from different data sources.

1. A method for providing control data for an ophthalmological laser of a treatment apparatus for correcting an astigmatism, wherein the method comprises the following steps performed by a control device:
determining first astigmatism values from predetermined visual disorder data of a patient, wherein the visual disorder data provides a measured refraction of the patient;
determining second astigmatism values from predetermined measurement data of a cornea of the patient, wherein the measurement data provides a morphology of the cornea;
ascertaining astigmatism correction data for astigmatism correction of the cornea, wherein the first and second astigmatism values are combined by a combination rule for ascertaining the astigmatism correction data; and
providing the control data for the ophthalmological laser, which includes the ascertained astigmatism correction data.

2. The method according to claim 1, wherein a difference between the first and second astigmatism values is determined, and wherein the combination rule is provided depending on the difference.

3. The method according to claim 1, wherein the first and second astigmatism values are divided into two vector components, wherein the first astigmatism values comprise a vector J_{0,R} for a cardinal astigmatism, in which cylinder axes of the astigmatism for 0° and 90° are perpendicular to each other, and a vector J_{45,R} for an oblique astigmatism, in which cylinder axes for 450 and 135° are perpendicular to each other, wherein the second astigmatism values comprise a corresponding vector J_{0,K} for the cardinal astigmatism and a corresponding vector J_{45,K} for the oblique astigmatism.

4. The method according to claim 1, wherein a first combination rule is provided as the combination rule, by which an average value between the first and second astigmatism values is determined.

5. The method according to claim 3, wherein the first combination rule includes that the cardinal astigmatism is calculated by (J_{0,R}+J_{0,K})/2 and the oblique astigmatism by (J_{45,R}+J_{45,K})/2.

6. The method according to claim 4, wherein a difference between the first and second astigmatism values is determined, and wherein the first combination rule is used if the difference between the first and second astigmatism values is below a preset threshold value.

7. The method according to claim 3, wherein a second combination rule is provided as the combination rule, by which one of the first and second astigmatism values identical in sign, and having a magnitude closer to zero, is selected.

8. The method according to claim 7 wherein the second combination rule includes that the cardinal astigmatism is provided by a maximum of J_{0,R} and J_{0,K}, if both are less than zero, and by a minimum of J_{0,R} and J_{0,K}, if both are greater than zero, and by zero for all further cases, and wherein the oblique astigmatism is provided by a maximum of J_{45,R} and J_{45,K}, if both are less than zero, and by a minimum of J_{45,R} and J_{45,K} if both are greater than zero, and by zero for all further cases.

9. The method according to claim 7, wherein a difference between the first and second astigmatism values is determined, and wherein the second combination rule is used if the difference between the first and second astigmatism values is above a preset threshold value.

10. The method according to claim 3, wherein a third combination rule is provided as the combination rule, by which the first and second astigmatism values are combined such that the cardinal astigmatism is considered as higher than the oblique astigmatism for the astigmatism correction.

11. The method according to claim 10, wherein the third combination rule includes that the cardinal astigmatism is provided by a maximum of J_{0,R} and J_{0,K}, if both are less than zero, and by a minimum of J_{0,R} and J_{0,K} if both are greater than zero, and by zero for all further cases, and wherein the oblique astigmatism is calculated by (J_{45,R}+J_{45,K})/2.

12. The method according to claim 10, wherein a difference between the first and second astigmatism values is determined, and wherein the third combination rule is used if the difference between the first and second astigmatism values is above a preset first threshold value, and it is further determined that a residual astigmatism above a preset second threshold value remains in the cornea after the treatment.

13. The method according to claim 1, wherein the measured refraction is determined by a refractometer and/or a phoropter and/or an aberrometer.

14. The method according to claim 1, wherein the morphology of the cornea is determined by a topography device.

15. A method for controlling a treatment apparatus, wherein the method comprises the following steps:
providing control data for an ophthalmological laser of a treatment apparatus according to the method of claim 1, and
transferring the provided control data to the ophthalmological laser of the treatment apparatus.

16. A control device, which is configured to perform a respective method according to claim 1.

17. A treatment apparatus with at least one ophthalmological laser for configured to perform separation of a corneal volume with predefined interfaces of a human or animal eye by optical breakdown, and at least one control device according to claim 16.

18. (canceled)

19. A computer-readable medium for storing a computer program, the computer program comprising commands which cause a treatment apparatus to execute the method according to claim 1.

20. The method according to claim 14, wherein the topography device is a keratograph, and the tomography device is an optical coherence tomograph, and/or a Scheimpflug tomograph.
